(19) United States
(12) Patent Application Publication
(10) Pub. No.: US 2006/0160905 A1
(43) Pub. Date: Jul. 20, 2006

(54) Compositions and methods for inhibiting pain

(76) Inventor: Raymond J. Bergeron JR., Gainesville, FL (US)

Correspondence Address: MILES & STOCKBRIDGE PC, 1751 PINNACLE DRIVE, SUITE 500, MCLEAN, VA, 22102-3833, US

(21) Appl. No.: 11/036,130

(22) Filed: Jan. 18, 2005

(51) Int. Cl. A61K 31/137

(52) U.S. Cl. 514/649

(57) ABSTRACT

A method and pharmaceutical composition for ameliorating pain in a human or non-human mammal suffering therefrom based on an antinociceptive amine having the formula: 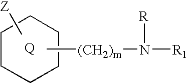
wherein: Z is H or OH, 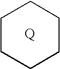 is phenyl or cyclohexyl,
R and R₁ are the same or different and may be H or linear or branched chain alkyl having 1 to 8 carbon atoms, m is an integer from 0 to 10, inclusive and wherein the amine exhibits a mean reduction in abdominal writhings exhibited by mice in the standard acetic acid-induced visceral pain mouse model of at least 20%; a mixture of the amines; a derivative, salt or complex of the amine wherein the derivative, salt or complex former is physiologically acceptable and the formation of said salt, derivative or complex does not materially affect the pain amelioration properties of the amine; a mixture of the derivatives, salts and/or complexes or a prodrug that provides the amine, mixture of the amines, the derivative, salt or complex, or a mixture of the derivatives, salts and/or complexes.

### BACKGROUND OF THE INVENTION

1. Field of the Invention

The present invention relates to methods and compositions for the amelioration of pain.

2. Description of the Prior Art

The opioids are a group of drugs, both natural and synthetic, that are employed primarily as centrally-acting analgesics and are opium or morphine-like in their properties (Gilman et al., 1980, GOODMAN AND GILMAN'S. THE PHARMACOLOGICAL BASIS OF THERAPEUTICS, Chapter 24:494-534, Pub. Pergamon Press; hereby incorporated herein by reference). The opioids include morphine and morphine-like homologs, including, e.g., the semisynthetic derivatives codeine (methylmorphine) and hydrocodone (dihydrocodeinone) among many other such derivatives. Morphine and related opioids exhibit agonist activity at the central nervous system or CNS (referring to the brain and spinal cord) (mu) opioid receptors as well as showing affinity for the delta and kappa opioid receptors, to produce a range of effects including analgesia, drowsiness, changes in mood and mental clouding. In addition to potent analgesic effects, the morphine-related opioids may also cause a number of undesirable side effects, including, for example, respiratory depression, nausea, vomiting, dizziness, mental clouding, dysphoria, pruritus, constipation, increased biliary tract pressure, urinary retention and hypotension. The development of tolerance to the opioid drugs and the risk of chemical dependence and abuse for these drugs is another undesirable side effect.

Morphine, which has been considered the prototypic opioid analgesic, has been available in many dosage forms, including immediate release oral dosage forms, and more recently, formulated into 12 hour controlled release formulations (e.g., MS Contin.RTM. tablets, commercially available from Purdue Frederick Company). Other opioid analgesics have been available as immediate release oral dosage forms, such as hydromorphone (e.g., Dilaudid.RTM., commercially available from Knoll Pharmaceuticals). More recently, another controlled release opioid analgesic, oxycodone, has become available (OxyContin.RTM., commercially available from Purdue Pharma). There are, of course, many other oral formulations of immediate release and sustained release opioids which are commercially available throughout the world.

The use of certain amines to alleviate pain is known in the prior art. For example, U.S. Pat. No. 6,710,087 discloses the treatment of neuropathic pain with sibutramine, [N-1-[1-(4-chlorophenyl)cyclobutyl]-3-methylbutyl]-N,N-dimethylamine]. It is disclosed in U.S. Pat. No. 6,376,553 to treat lower back pain with N,N-dimethyl-1-[1-(4-chlorophenyl)cyclobutyl]-3-methylbutyl amine hydrochloride. U.S. Pat. No. 5,688,830 indicates that 2-(2,6-dimethylphenoxy)-1-methylethyl-ethylamine is useful for the treatment of pain. Other US patents disclosing the use of monoamine compounds to alleviate pain include U.S. Pat. Nos. 6,642,257; 5,843,942 and 5,063,231.

It is an object of the invention to provide novel compositions and methods for the treatment of neuropathic pain involving the use of certain monoamines.

### SUMMARY OF THE INVENTION

One embodiment of the invention relates to a method for ameliorating pain in a human or non-human mammal suffering therefrom comprising administering to the mammal an effective amount of an antinociceptive amine having the formula: 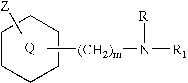
wherein: Z is H or OH, 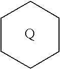
is phenyl or cyclohexyl,
R and R₁ are the same or different and may be H or linear or branched chain alkyl having 1 to 8 carbon atoms,
m is an integer from 0 to 10, inclusive
   and wherein the amine exhibits a mean reduction in abdominal writhings exhibited by mice in the standard acetic acid-induced visceral pain mouse model of at least 20%; a mixture of the amines; a derivative, salt or complex of the amine wherein the derivative, salt or complex former is physiologically acceptable and the formation of said salt, derivative or complex does not materially affect the pain amelioration properties of the amine; a mixture of the derivatives, salts and/or complexes or a prodrug that provides the amine, mixture of the amines, the derivative, salt or complex, or a mixture of the derivatives, salts and/or complexes

Another embodiment of the invention concerns a composition for the for ameliorating pain in a human or non-human mammal suffering therefrom comprising (1) an effective amount of an antinociceptive amine having the formula: 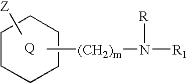
wherein: Z is H or OH, 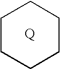
   is phenyl or cyclohexyl,
R and R₁ are the same or different and may be H or linear or branched chain alkyl having I to 8 carbon atoms,
m is an integer from 0 to 10, inclusive
   and wherein the amine exhibits a mean reduction in abdominal writhings exhibited by mice in the standard acetic acid-induced visceral pain mouse model of at least 20%; a mixture of the amines; a derivative, salt or complex of the amine wherein the derivative, salt or complex former is physiologically acceptable and the formation of said salt, derivative or complex does not materially affect the pain amelioration properties of the amine; a mixture of the derivatives, salts and/or complexes or a prodrug that provides the amine, mixture of the amines, the derivative, salt or complex, or a mixture of the derivatives, salts and/or complexes and (2) a pharmacologically acceptable carrier therefore.

Other objects, features and advantages of the present invention will become apparent to those skilled in the art from the following detailed description. It should be understood, however, that the detailed description and specific examples, while indicating preferred embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 sets forth the structural formulas of 6 monoamines tested for their effectiveness as pain-relief agents.

FIGS. 2 and 3 set forth reaction schemes for the preparation of monoamines described in the application.

FIGS. 4-10 set forth the results of the standard acetic acid-induced visceral pain mouse test employing various of the monoamines described herein.

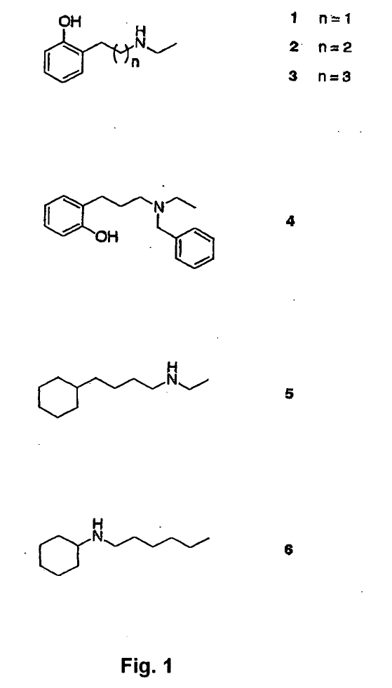

### DETAILED DESCRIPTION OF THE INVENTION

The present invention is predicated on the discovery that certain of the monoamines having the structure: 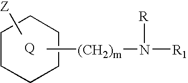
wherein: Z is H or OH, 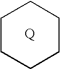
is phenyl or cyclohexyl,
R and R₁ are the same or different and may be H or linear or branched chain alkyl having 1 to 8 carbon atoms,
m is an integer from 0 to 10, inclusive
   and wherein the amine exhibits a mean reduction in abdominal writhings exhibited by mice in the standard acetic acid-induced visceral pain mouse model of at least 20%; a mixture of the amines; a derivative, salt or complex of the amine wherein the derivative, salt or complex former is physiologically acceptable and the formation of said salt, derivative or complex does not materially affect the pain amelioration properties of the amine; a mixture of the derivatives, salts and/or complexes or a prodrug that provides the amine, mixture of the amines, the derivative, salt or complex, or a mixture of the derivatives, salts and/or complexes effectively ameliorate the effects of neuropathic pain.

By merely subjecting selected amines having the above structure to the standard acetic acid-induced visceral pain mouse model and determining that the amine exhibits a mean reduction in abdominal writhings by 20%, effective amines can readily be identified.

The acetic acid-induced visceral pain mouse model test is, of course, well known to those skilled in the art and is described in the seminal paper by Collier et al [Collier, H. O. J., L. C. Dinneen, C. A. Johnson and C. Schneider 1968. The abdominal constriction response and its suppression by analgesic drugs in the mouse. Br. J Pharmacol. Chemother 32: 295; see also, S. Z. Idid et al, ASEAN Review of Biodiversity and Environmental Conservation (ARBEC), May 1998, Article IV, May 1998, entitled, “Evaluation of Analgesia Induced by Mitragynine, Morphine and Paracetamol on mice”].

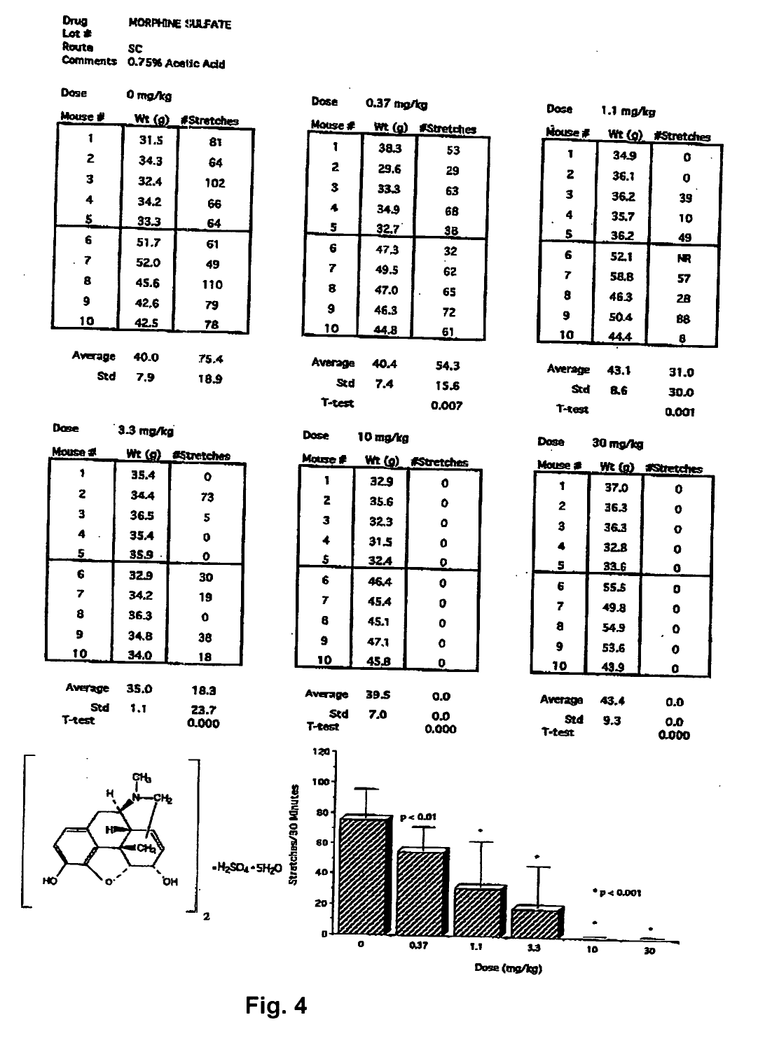

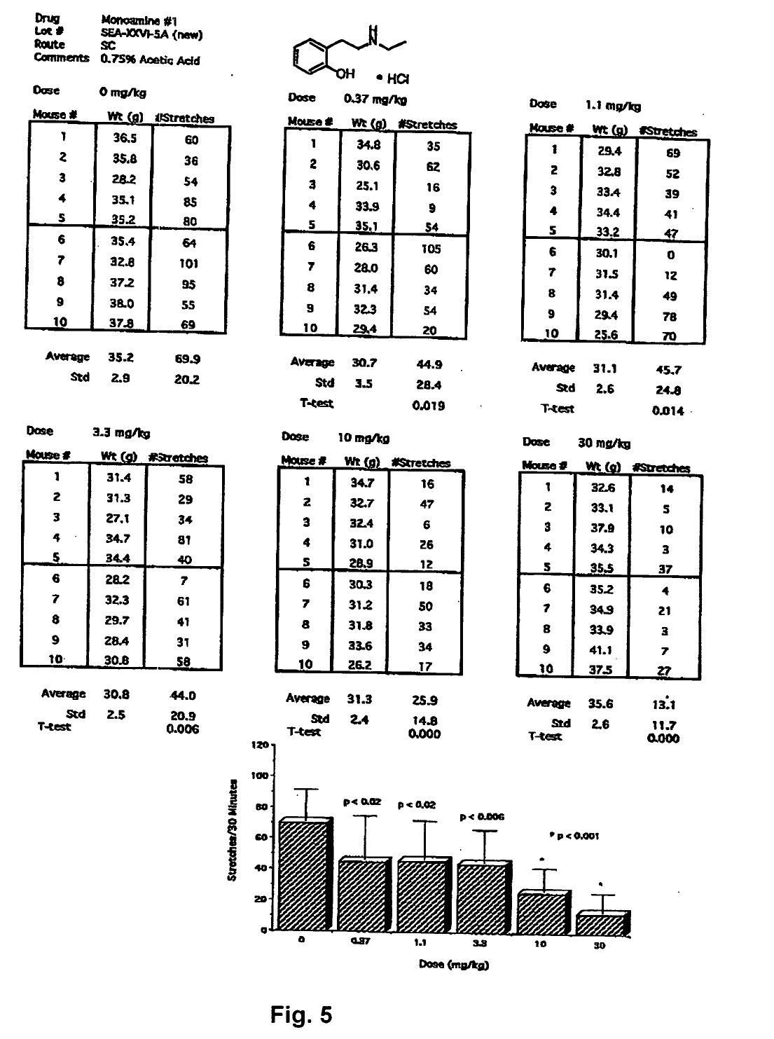

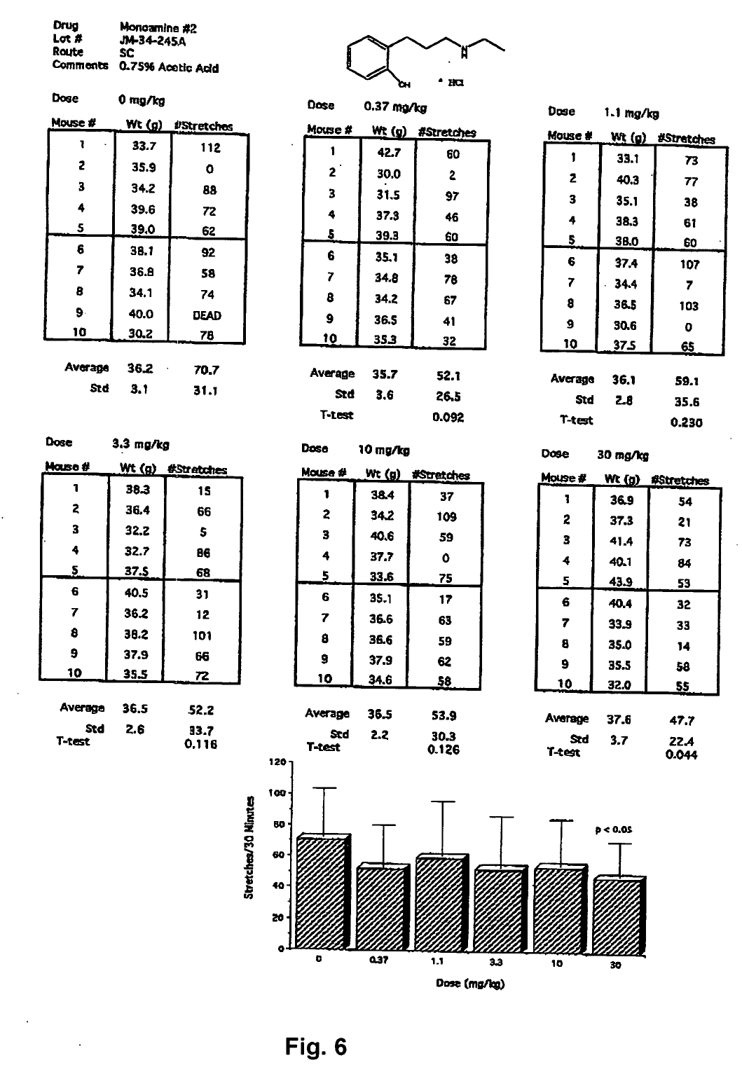

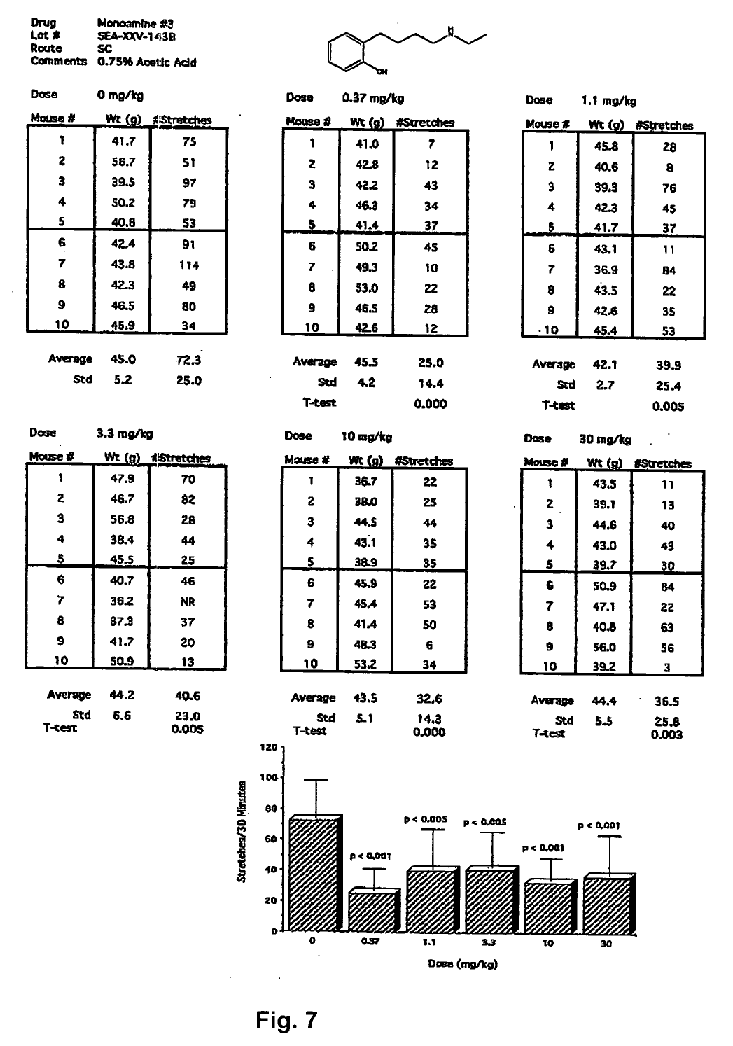

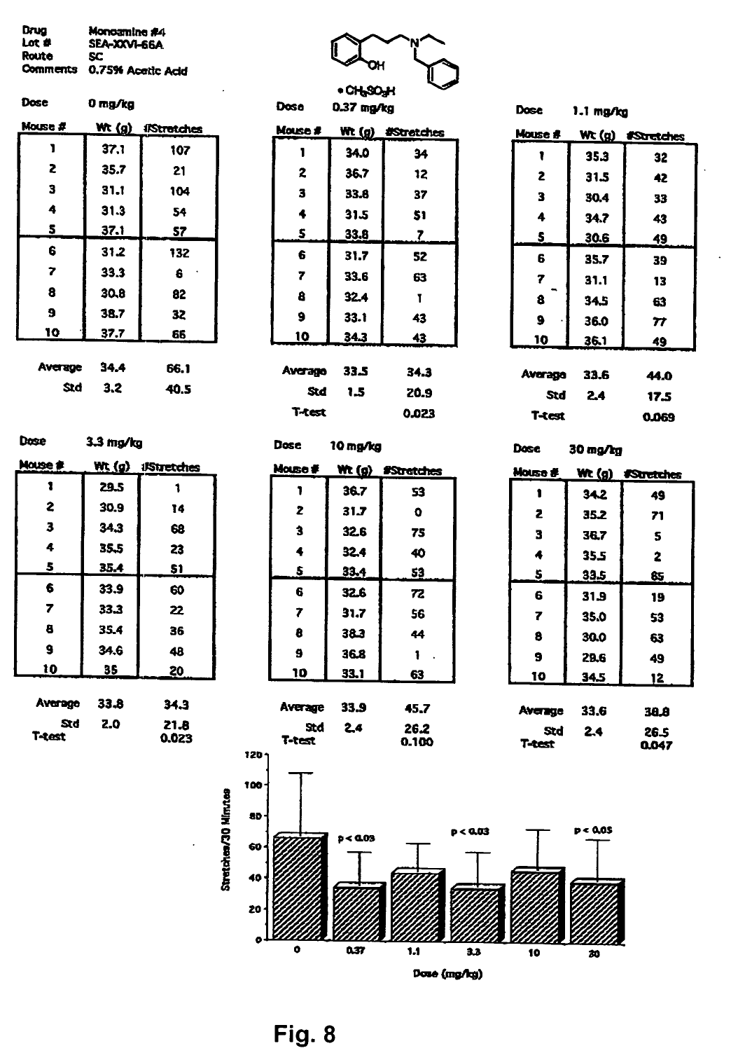

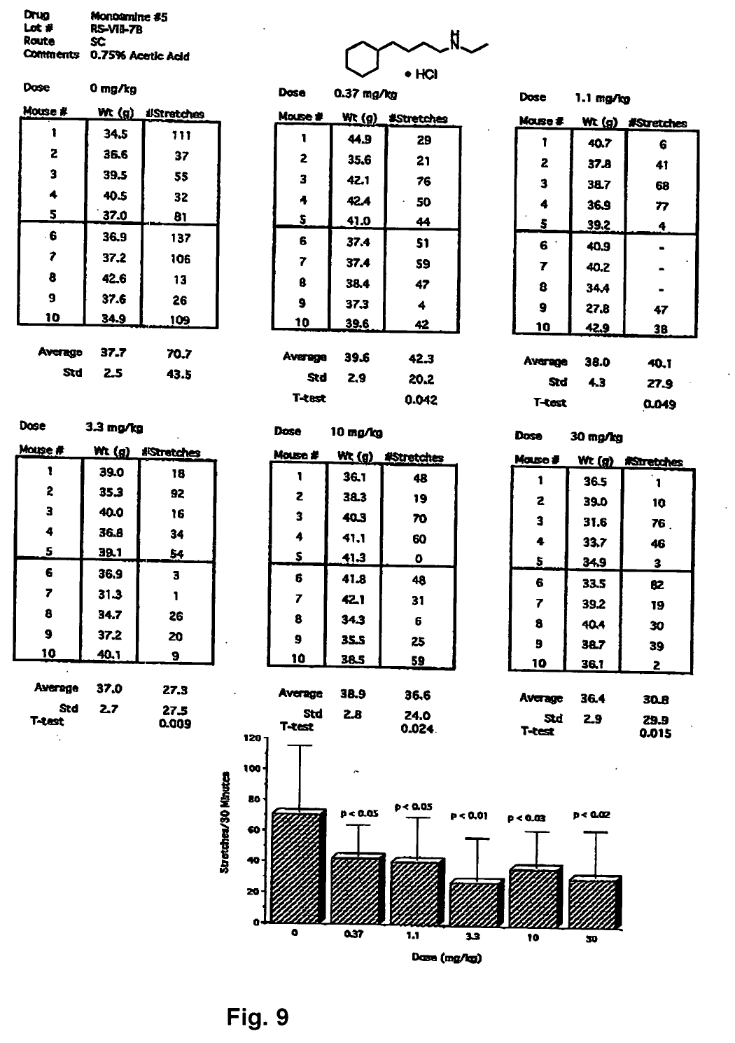

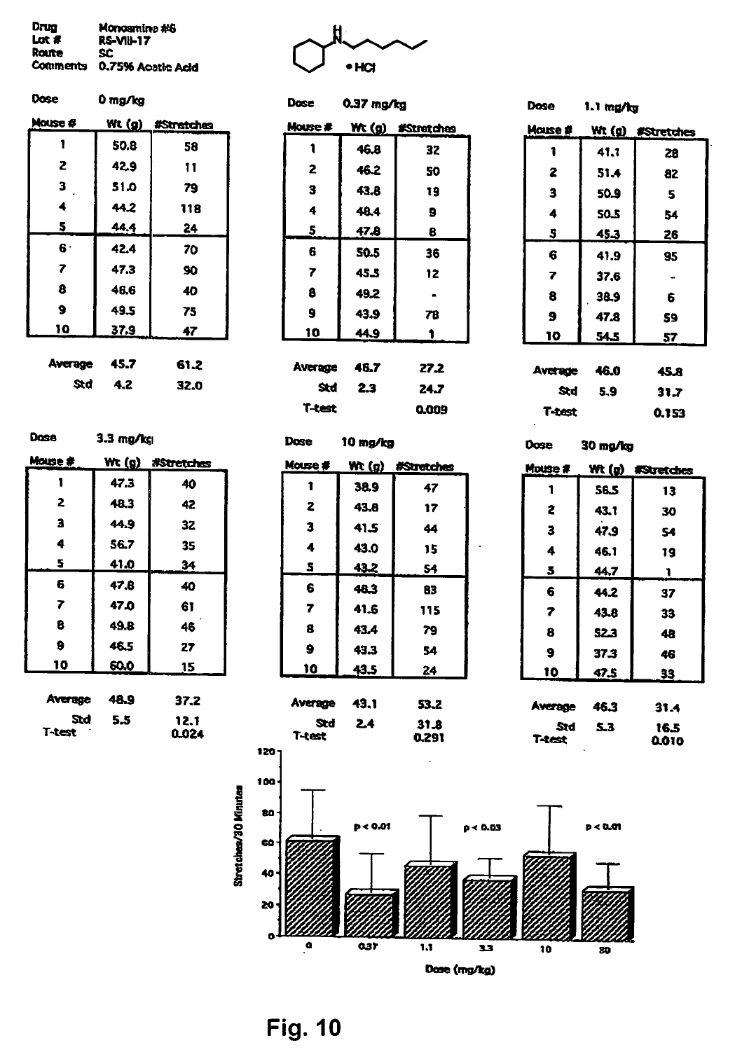

Several different monamines defined by the above structural formula were evaluated in the acetic acid-induced visceral pain mouse model to demonstrate the efficacy of the compounds in inhibiting pain. The drugs are identified below as Monoamine #1-6. Five groups of five mice (male Harlan ICR, weight 35-50 g) were used to test each of the drugs. Control animals were tested as part of each experiment as well. The mice in each group were weighed and identified with a tail marking and were given a single dose of one of the drugs at 1 ml/100 g SC. The first group served as controls and was given only saline; the other five groups were given a drug at five doses ranging from 0.37 mg/kg up to 30.0 mg/kg. After the drug was administered, the mice were allowed to acclimate for one-half hour and were then given an IP injection of 0.75% Glacial acetic acid at a volume of 1 ml/100 g. Once the acid was injected, the mice were put into individual clear containers for observation. The mice were then watched continuously for one-half hour and the number of “stretches” or writhes were counted. The animal was considered to be stretching when its front legs stepped forward and at least one hind leg was extended out behind them. At the end of the thirty-minute observational period the mice were sacrificed with CO₂. The same procedure was followed for all of the different compounds examined. The results of the tests are set forth in FIGS. 4-10.

In order to demonstrate the effectiveness of the monoamines, it is important to know that the control mice typically stretch 65-70 times/30 minutes. However, mice that were dosed with 10 mg/kg of Monoamine #1 only writhed 25.9±14.8 times (P<0.001 vs. controls). The number of stretches decreased further at a dose of 30 mg/kg, with 13.1±11.7 stretches/30 minutes (P<0.001 vs. controls). The P-values show a statistical significance at all doses tested, confirming that there is an effect on inhibiting visceral pain at 0.37 mg/kg that continues through 30 mg/kg.

Groups of mice were injected with Monoamine #2 at doses ranging from 0.37 mg/kg to 30.0 mg/kg. The mice that were dosed with 30 mg/kg writhed 47.7±22.4 times (P<0.05) versus controls, showing that this drug had only a minimal effect on inhibiting visceral pain. Several doses of Monoamine #3 were administered to five groups of mice in hopes of eliciting a nociceptive response. The lowest dose was 0.37 mg/kg and the highest was 30.0 mg/kg. All of the doses were found to result in significantly fewer stretches than the control mice, indicating that this drug also has an effect on visceral pain.

Multiple doses of Monoamine #4 were administered to five groups of mice. The lowest dose was 0.37 mg/kg and the highest was 30.0 mg/kg. Although several doses of the test drug resulted in fewer stretches than those observed in the control group, the number of stretches in these groups were within error of those of groups that were not significant, suggesting that the compound is not effective against visceral pain.

Several doses of Monoamine #5 were administered to five groups of mice in hopes of eliciting a nociceptive response. The lowest dose was 0.37 mg/kg and the highest was 30.0 mg/kg. All of the doses were found to result in significantly fewer stretches than the control mice indicating that this drug also has an effect on visceral pain.

Lastly, multiple doses of Monoamine #6 were administered to five groups of mice. The lowest dose was 0.37 mg/kg and the highest was 30.0 mg/kg. Although several doses of the test drug resulted in fewer stretches than those observed in the control group, the number of stretches in these groups were within error of those of groups that were not significant, suggesting that the compound is not effective against visceral pain.

In order to put these findings into perspective, morphine was used as a positive control. It was administered to the mice at doses ranging from 0.37 mg/kg to 30.0 mg/kg SC. The most effective doses for the morphine were 10.0 or 30 mg/kg at which point the mice completely stopped writhing, i.e., the average was 0.

In conclusion, three of the six monoamines evaluated had a significant effect on reducing acetic acid-induced visceral pain. Monoamines #1, #3 and #5, like morphine, were effective at all of the doses tested. Finally, Monoamines #2, #4 and #6 had little/no effect on the inhibition of pain in this model.

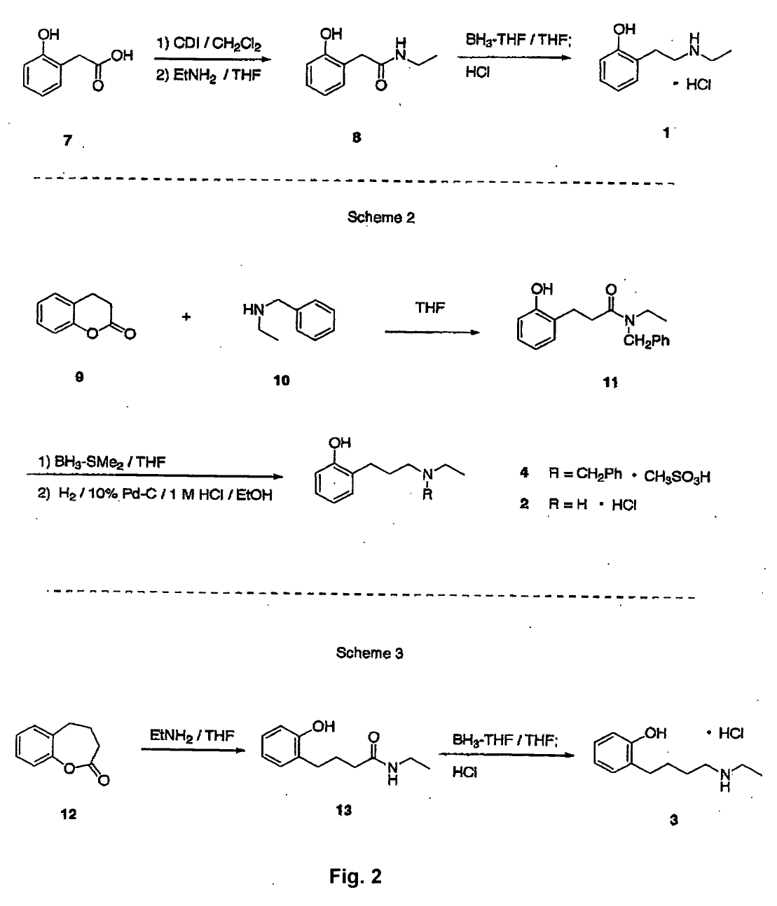

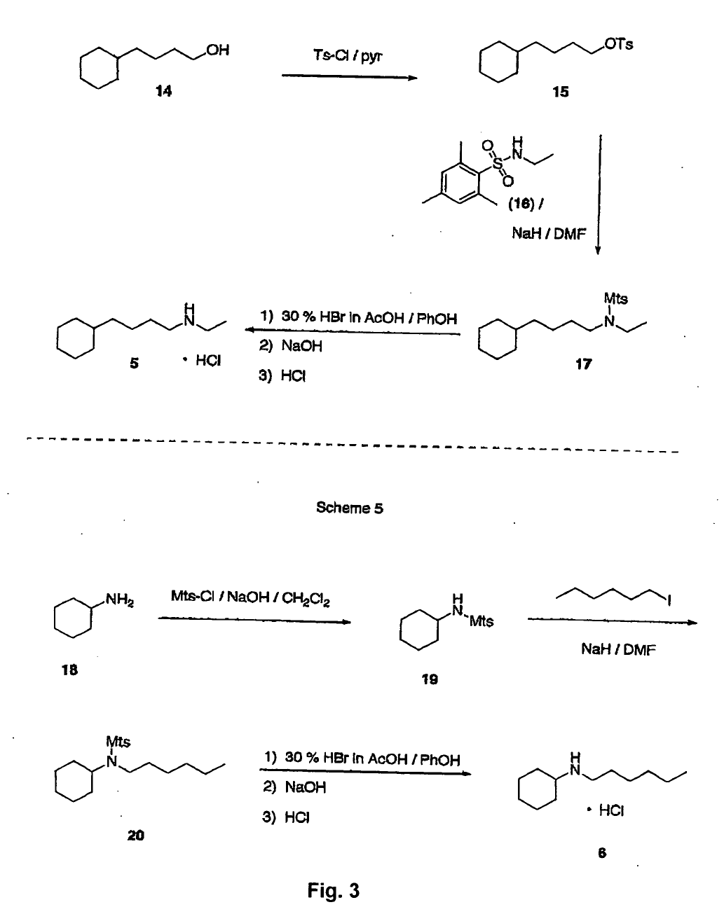

Monoamines 1-6 as their hydrochloride or methanesulfonate salts were synthesized by the methodology of Schemes 1-5 in FIGS. 2 and 3.

### EXAMPLE 1

2-Hydroxyphenylacetic acid (7) was activated using carbonyldiimidazole (CDI) in CH₂Cl₂; treatment with a 20% excess of ethylamine in THF afforded 8. Reduction of amide 8 with borane-tetrahydrofuran complex in THF and acidification gave the secondary amine salt 1. (Scheme 1, FIG. 2). 2-Hydroxy-N-ethylbenzeneacetamide (8). CDI (5.86 g, 36.1 mmol) was added to a solution of 7 (5.0 g, 33 mmol) in CH₂Cl₂ (40 mL) under nitrogen. After 30 min. of stirring, the reaction solution was cooled in an ice bath, and ethylamine (2 M in THF, 20 mL, 40 mmol) was added dropwise over 30 min. After stirring for 18 h at room temperature the reaction was quenched with H₂O (50 mL) and 1 N HCl (25 mL), the layers were separated, and the aqueous phase extracted further with CH₂Cl₂ (2×50 mL). The organic extracts were washed with H₂O (50 mL) and saturated NaCl (50 mL, dried over Na₂SO₄, and concentrated in vacuo. Purification by flash column chromatography using silica gel 32-63 (1:1 hexanes/EtOAc) produced 1.8 g (31%) of 8 as a pale yellow solid: ¹H NMR (CDCl₃) δ 1.15 (t, 3 H, J=7.2), 3.24-3.38 (m, 2 H), 3.54 (s, 2 H), 6.00 (br s, 1 H), 6.78-6.86 (m, 1 H), 6.95-7.02 (m, 2 H), 7.15-7.22 (m, 1H), 9.99 (br s, 1 H).

### EXAMPLE 2

Ring opening [Bussolari et al., Tetrahedron Letters 40 (1999) 1241-1244] of dihydrocoumarin (9) with N-ethylbenzylamine (10) in THF at room temperature led to carboxamide 11. Heating 11 with borane-methyl sulfide complex in THF generated tertiary amine 4, which was transformed into its water soluble mesylate salt. Free amine 11 was debenzylated under mild catalytic conditions (Pd-C, dilute HCl, EtOH), yielding amine 2 as its HCl salt (Scheme 2, FIG. 2). 2-[2-(Ethylamino)ethyl]phenol Hydrochloride (1). Borane-tetrahydrofuran complex (1 M in THF, 40 mL, 40 mmol) was added to a solution of 8 (1.7 g, .9.5 mmol) in distilled THF (60 mL). After 64 h of stirring at room temperature under nitrogen, the reaction mixture was quenched with EtOH (10 mL) and concentrated in vacuo. The residue was taken up in 6 N HCl (100 mL), heated at reflux for 2 h, and concentrated in vacuo. Sodium hydroxide (2 N, 10 mL and 20%, 10 mL) was added, followed by extraction with CHCl₃ (4×50 mL). The organic extracts were dried over Na₂SO₄ and concentrated by rotary evaporation. The concentrate was treated with EtOH (50 mL) and concentrated HCl (10 mL), and volatiles were removed under reduced pressure. Recrystallization with 40% EtOH/toluene generated 1.2 g (63%) of 1 as a white solid, mp 168-170° C.: ¹H NMR (D₂O) δ 1.27 (t, 3 H, J=7.4), 3.01 (t, 2 H, J=7.2), 3.11 (q, 2 H, J=7.3), 3.30 (t, 2 H, J=7.2), 6.93-7.00 (m, 2 H), 7.22-7.29 (m, 2 H). Anal. Calcd for C₁₀H₁₆ClNO: C, 59.55; H, 8.00; N, 6.94. Found: C, 59.55; H, 8.16; N, 6,92.

### EXAMPLE 3

4,5-Dihydro-1-benzoxepin-2(3H)-one (12) [Cambie et al., Australian Journal of Chemistry (1998), 51(12), 1167-1174] was also subjected to ethylamine-induced ring cleavage, giving 13. Borane reduction of amide 13 and acidification furnished the secondary amine 3 as its HCl salt (Scheme 3, FIG. 2).

### EXAMPLE 4

4-Cyclohexyl-1-butanol (14) was converted to tosylate 15, which was used to alkylate N-ethylmesitylenesulfonamide (16) [Bergeron et al., J. Med. Chem. (1994), 37, 3464-3476]. Deprotection of adduct 17 was accomplished with. 30% HBr in HOAc and phenol, resulting in N-(4-cyclohexylbutyl)-N-ethylamine 5, isolated as its HCl salt (Scheme 4, FIG. 3).

### EXAMPLE 5

Cyclohexylamine (18) was derivatized as sulfonamide 19, which was heated with 1-iodohexane (NaH, DMF) to give 20. Removal of the amine protecting group of 20 provided N-hexylcyclohexanamine 6 [Ganguly et al., polyhedron (1990), 9(20), 2517-26; Yoon et al., Synthetic Communications, (1993), 23(11), 1595-9] as its HCI salt (Scheme 5, FIG. 3).

1. A method for ameliorating pain in a human or non-human mammal suffering therefrom comprising administering to the mammal an effective amount of an antinociceptive amine having the formula: 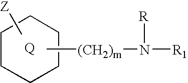
wherein: Z is H or OH, 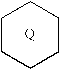
is phenyl or cyclohexyl,
R and R₁ are the same or different and may be H or linear or branched chain alkyl having 1 to 8 carbon atoms,
m is an integer from 0 to 10, inclusive
and wherein the amine exhibits a mean reduction in abdominal writhings exhibited by mice in the standard acetic acid-induced visceral pain mouse model of at least 20%; a mixture of the amines; a derivative, salt or complex of the amine wherein the derivative, salt or complex former is physiologically acceptable and the formation of said salt, derivative or complex does not materially affect the pain amelioration properties of the amine; a mixture of the derivatives, salts and/or complexes or a prodrug that provides the amine, mixture of the amines, the derivative, salt or complex, or a mixture of the derivatives, salts and/or complexes.

2. The method of claim 1 wherein said ring is cyclohexyl.

3. The method of claim 1 wherein said ring is phenyl.

4. The method of claim 1 wherein said ring is phenyl and Z is OH.

5. The method of claim 1 wherein said ring is cyclohexyl and Z is H.

6. The method of claim 1 wherein said amine has the formula: 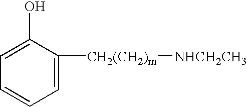
wherein: m is an integer from 1 to 3, inclusive.

7. The method of claim 6 wherein m is 1.

8. The method of claim 6 wherein m is 2.

9. The method of claim 1 wherein said amine has the formula:

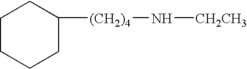

10. The method of claim 1 wherein said amine has the formula:

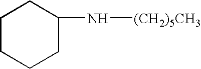

11. A composition for ameliorating pain in a human or non-human mammal suffering therefrom comprising (1) an effective amount of an antinociceptive amine having the formula: 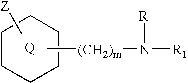
wherein: Z is H or OH, 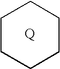
is phenyl or cyclohexyl,
R and R₁ are the same or different and may be H or linear or branched chain alkyl having 1 to 8 carbon atoms,
m is an integer from 0 to 10, inclusive
and wherein the amine exhibits a mean reduction in abdominal writhings exhibited by mice in the standard acetic acid-induced visceral pain mouse model of at least 20%; a mixture of the amines; a derivative, salt or complex of the amine wherein the derivative, salt or complex former is physiologically acceptable and the formation of said salt, derivative or complex does not materially affect the pain amelioration properties of the amine; a mixture of the derivatives, salts and/or complexes or a prodrug that provides the amine, mixture of the amines, the derivative, salt or complex, or a mixture of the derivatives, salts and/or complexes and (2) a pharmacologically acceptable carrier therefore.

12. The composition of claim 11 wherein said ring is cyclohexyl.

13. The composition of claim 11 wherein said ring is phenyl.

14. The composition of claim 11 wherein said ring is phenyl and Z is OH.

15. The composition of claim 11 wherein said ring is cyclohexyl and Z is H.

16. The composition of claim 11 wherein said amine has the formula: 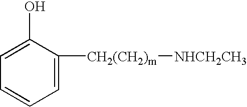
wherein: m is an integer from 1 to 3, inclusive.

17. The composition of claim 16 wherein m is 1.

18. The composition of claim 16 wherein m is 2.

19. The composition of claim 11 wherein said amine has the formula:

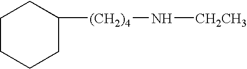

20. The composition of claim 11 wherein said amine has the formula:

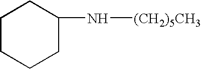

21. An article of manufacture comprising packaging material and a pharmaceutical agent contained within said packaging material, wherein said pharmaceutical agent is effective for the treatment of a subject suffering from pain, and wherein said packaging material comprises a label which indicates that said pharmaceutical agent can be used for ameliorating the symptoms associated with pain, and wherein said pharmaceutical agent has the formula: 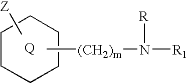
wherein: Z is H or OH, 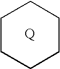
is phenyl or cyclohexyl,
R and R₁ are the same or different and may be H or linear or branched chain alkyl having 1 to 8 carbon atoms,
m is an integer from 0 to 10, inclusive
and wherein the amine exhibits a mean reduction in abdominal writhings exhibited by mice in the standard acetic acid-induced visceral pain mouse model of at least 20%; a mixture of the amines; a derivative, salt or complex of the amine wherein the derivative, salt or complex former is physiologically acceptable and the formation of said salt, derivative or complex does not materially affect the pain amelioration properties of the amine; a mixture of the derivatives, salts and/or complexes or a prodrug that provides the amine, mixture of the amines, the derivative, salt or complex, or a mixture of the derivatives, salts and/or complexes.

22. An amine having the formula: 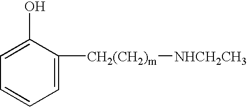
wherein: m is an integer from 1 to 3, inclusive.

23. An amine having the formula:

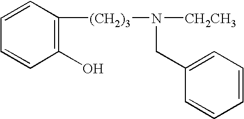

24. An amine having the formula: [chemical structure]
